(19) United States
(12) Patent Application Publication
(10) Pub. No.: US 2023/0285429 A1
(43) Pub. Date: Sep. 14, 2023

(54) CANNABINOID ACID FORMULATIONS, DELIVERY SYSTEMS, AND METHODS OF USE

(71) Applicant: Flora Growth U.S. Holdings Corp., (US)

(72) Inventor: Annabelle Manalo-Morgan, Franklin, TN (US)

(21) Appl. No.: 18/104,760

(22) Filed: Feb. 1, 2023

Related U.S. Application Data
(60) Provisional application No. 63/305,448, filed on Feb. 1, 2022.

(51) Int. Cl. A61K 31/00; A61M 21/02; A61K 47/14

(52) U.S. Cl. CPC A61K 31/658; A61M 21/02; A61K 47/14; A61M 2021/0027

(57) ABSTRACT

A method for treating illness utilizing a neurofeedback therapy and cannabinoid formulation. The method includes providing a composition having a triglyceride comprising a short-chain fatty acid, an active ingredient comprising a cannabinoid acid which may include a cannabinoid acid selected from the group consisting of CBDA, CBGA, and CBCA. The method may also include delivering the formulation to the patient via a delivery system wherein at least 50% of the cannabinoid acid is preserved in its acidic form at a target site. The neurofeedback therapy may include alpha entrainment wherein alpha brain waves of the patient are increased in amplitude.

### CROSS-REFERENCES TO RELATED APPLICATIONS

This application claims the benefit of priority to U.S. Provisional Patent Application No. 63/305,448 filed Feb. 1, 2022, the contents of which are hereby incorporated by reference in their entirety.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### TECHNICAL FIELD

The present invention relates generally to cannabinoid formulations including cannabinoids as active ingredients for preventative and therapeutic use, delivery systems for the formulations, and further to methods of treatment.

More particularly, this invention pertains to cannabinoid formulations including fatty acids, in particular short chain triglycerides, and to methods of treatment using the formulations in combination with additional therapeutic interventions.

### BACKGROUND

Chronic pain (lasting more than 3 months) can be one of the most difficult conditions to treat in patients. In particular, chronic pain is a complex and multidimensional condition that is affected by a patient's unique biology, mood, social environment, and past experiences. Because of the challenges with providing effective therapies for pain management, clinical practitioners have searched for effective and safer alternatives to opioids to alleviate pain without the harmful effects and addiction. Cannabis, and more particularly CBD (cannabidiol), has emerged as a promising pharmaceutical agent to treat pain, inflammation, seizures, and anxiety.

The acidic forms of cannabinoid acids have been overshadowed by the multitude of cannabinoids and the study of their various effects on the human body. As is well known, cannabinoids come from the precursor molecule, cannabigerolic acid (CBGA), also dubbed “the mother of all cannabinoids.” Plant enzymes may then convert CBGA into three major cannabinoid precursor compounds, tetrahydrocannabinolic acid (THCA), cannabichromenic acid (CBCA), and cannabidiolic acid (CBDA). Additionally, other precursor cannabinoid acids are produced including cannabinolic acid (CBNA) and cannabicyclolic acid (CBLA). Some cannabinoids, such as cannabidiol (CBD), result from the decarboxylation of the precursor compounds. While cannabinoids have received most of the focus in research, recent studies have suggested that cannabinoid acids may have physiologically relevant effects.

However, one major issue in the use of cannabinoid acids for preventative and therapeutic use is the poor bioavailability and delivery systems for the acidic forms. Little information is available in the literature on delivery systems that can preserve the acidic forms of cannabinoid acids while also providing high bioavailability. And the underlying chemical interactions that could lead to therapeutically effective formulations of cannabinoid acids are not well understood.

The understanding of the role of CBD in pain management continues to evolve, and literature has shown that CBD may provide its pain-relieving therapies through various interactions and modulation of the endocannabinoid, inflammatory, and nociceptive (pain sensing) systems. It is well appreciated that the endocannabinoid system consists of cannabinoid receptors that interact with our own naturally occurring cannabinoids. Generally, this system is involved in regulating metabolism and appetite, mood and anxiety, and pain perception, among other functions as well.

With respect to pain perception, there is very little research on the effects of CBD on brainwaves of those suffering from chronic pain. Existing research on the impact of CBD on an EEG is still not well elucidated. No research has been found to date exploring the effect of CBD on the EEG of chronic pain (lasting more than 3 months) patients. Some research has found that the brain plays an important role in how much pain people feel and whether they develop chronic pain symptoms. It is not well understood what the physiological, emotional, chemical, and/or molecular differences, if any, are between people and the amount of pain they fell to a stimulus, or whether they develop chronic pain. Further, it is not well understood ways to effectively treat such chronic pain. Thus, ways to more effectively measure and/or treat changes in the brain that may contribute to chronic pain are needed. Additionally, formulations and delivery systems sufficient to effectively and efficiently deliver, and preserve upon delivery, the acidic forms of cannabinoid acids and allow for high bioavailability of the molecules is needed to help treat chronic pain. Such aspects are disclosed herein.

### BRIEF SUMMARY

In a first aspect, a formulation including a cannabinoid acid is provided. In one embodiment of the first aspect, the cannabinoid acid is CBGA. In another embodiment of the first aspect, the cannabinoid acid is CBDA. In yet another embodiment of the first aspect, the cannabinoid acid is CBCA. In another embodiment of the first aspect, the cannabinoid acid is THCA. In yet another embodiment of the first aspect, the cannabinoid acid is a combination of one or more of CBGA, CBDA, CBCA, and THCA.

In an embodiment of the first aspect, the formulation may include a cannabinoid acid and moringa leaf extract. In one embodiment of the first aspect, the formulation may include a cannabinoid acid and a dietary supplement. In certain embodiments, the dietary supplement may be a vitamin, mineral, herb, botanical, probiotic, amino acid, enzyme, antioxidants, and the like. In some embodiments, the dietary supplement may be Vitamin A, Vitamin B, Vitamin C, Vitamin D, Vitamin E, Vitamin K. In some embodiments, the formulation may include a cannabinoid acid and other additives. In certain embodiments, the additives may include magnesium oxide and/or zinc gluconate. In some embodiments of the first aspect, the formulation may include any combination of the foregoing.

In an embodiment of the first aspect, the formulation may include a cannabinoid acid and a preservative. In certain embodiments, the preservative may include one or more of antioxidants, such as Vitamin C, Vitamin E, butylatedhydroxyanisole, butylatedhydroxytoluene, propyl gallate, and the like; antimicrobial agents, such as phenol, parabens, aryl and alkyl acids, and the like. In some embodiments, the formulations may include a combination of the preservatives as well as a combination of the preservatives and any combination of other formulations disclosed herein.

In one embodiment of the first aspect, the formulation comprises CBGA, moringa leaf extract, ascorbic acid, magnesium oxide, and zinc gluconate. In another embodiment of the first aspect, the formulation comprises CBDA, moringa leaf extract, ascorbic acid, magnesium oxide, and zinc gluconate. And in yet another embodiment of the first aspect, the formulation comprises CBGA, CBDA, moringa leaf extract, ascorbic acid, magnesium oxide, and zinc gluconate.

In an embodiment of the first aspect, the formulation may include an active ingredient. In certain embodiments, the active ingredient may include a cannabinoid acid. In another embodiment, the active ingredient may include one or more of a cannabinoid acid. In one embodiment, the formulation may include an active ingredient that is not a cannabinoid acid. In some embodiments, the active ingredient may be a polarizing active ingredient. It will be further understood that the formulation may include one or more active ingredients.

In an embodiment of the first aspect, the formulation may include a fatty acid chain. In some embodiments, the fatty acid chain is at least 10 carbons in length, is at least 12 carbons in length, is at least 14 carbons in length, at least 16 carbons in length, at least 18 carbons in length, at least 20 carbons in length, at least 22 carbons in length, or is at least 24 carbons in length. In some embodiments, the fatty acid chain is at least 14 carbons in length. In certain embodiments, the formulation may include a short fatty acid chain wherein the fatty acid chain is from 0-6 carbons in length.

In an embodiment of the first aspect, a cannabinoid acid may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBDA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBGA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBCA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, a combination of CBDA and CBGA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, a combination of CBDA and CBCA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, a combination of CBCA and CBGA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In a second aspect, a delivery system is described for delivering a formulation comprising at least an acidic form of cannabinoid acid.

In one embodiment of the second aspect, the delivery system is selected from oral, buccal, sublingual, topical, inhalation, rectal, or injection delivery.

In one embodiment of the second aspect, the delivery system is selected from oral, buccal, or sublingual.

In one embodiment of the second aspect, the delivery system is configured to preferentially transport at least a portion of the formulation to the lymphatic system.

In one embodiment of the second aspect, the delivery system may include a fatty acid chain. In some embodiments, the fatty acid chain is at least 10 carbons in length, is at least 12 carbons in length, is at least 14 carbons in length, at least 16 carbons in length, at least 18 carbons in length, at least 20 carbons in length, at least 22 carbons in length, or is at least 24 carbons in length. In some embodiments, the fatty acid chain is at least 14 carbons in length. In certain embodiments, the formulation may include a short fatty acid chain wherein the fatty acid chain is from 0-6 carbons in length.

In a third aspect, a method of treating a disease or medical condition is disclosed.

In some embodiments, a formulation comprising one or more cannabinoid acids may be useful in treating and/or preventing certain diseases or medical conditions.

In one embodiment of the third aspect, CBGA may have analgesic, antibacterial, antiviral, anti-inflammatory, and anti-proliferative properties. Additionally, CBGA may combat cardiovascular disease, metabolic disorders, and certain cancers.

In another embodiment of the third aspect, CBDA may have physiological benefits including treatment of inflammation, anxiety, seizures, certain cancers, pain, and nausea. CBDA may also demonstrate anti-bacterial and anti-viral properties.

In one embodiment of the third aspect, a method of treating or preventing viral infections may be disclosed. In certain embodiments, a formulation comprising a cannabinoid acid(s) may be used to treat or prevent viral infections through modulating the immune response, the immune system, viral recognition and entry to target cells, viral replication, viral maturation, and/or viral release.

In some embodiments of the third aspect, a method of treatment or prevention is disclosed utilizing immunomodulation pathways, including, but not limited to pro- and anti-inflammatory functions, Cox-2 pathways, T-cell pathways, neutrophil responses. It will be appreciated that other immunomodulation pathways, cell types, and molecules involved in the host immune response may be affected by the disclosed formulations herein and are not to be limited in the scope of this disclosure.

In an embodiment of the third aspect, a method of treating or preventing infection of SARS-CoV-2 is disclosed. In certain embodiments, inventors propose a mechanism of action contributing to antiviral activity of SARS-CoV-2 via immunomodulation and specific formulations comprising one or more cannabinoid acids. In some embodiments, the formulation of one or more cannabinoid acids may be more effective against particular variants of SARS-CoV-2.

### DETAILED DESCRIPTION

The following description and examples illustrate some exemplary embodiments of the disclosure in detail. Those of skill in the art will recognize that there are numerous variations and modifications of the embodiments in this disclosure that are encompassed by its scope. Accordingly, the description of a certain exemplary embodiment should not be deemed to limit the scope of the various embodiments described herein.

To facilitate an understanding of the disclosed embodiments, certain terms and definitions are provided below:

The term “active ingredient” as used herein may be understood to refer to a component(s) that provides pharmacological activity or other direct effect in the diagnosis, cure, mitigation, treatment, or prevention of a disease or disorder, or to affect the structure or any function of the body of a person or animal.

The term “bioavailability” as used herein may be understood to refer to the fraction or dose of an administered substance capable of being absorbed by the body's circulatory system and available for use or storage by the body. Bioavailability may be measured by calculating the concentration of a substance administered to an individual's body over time. Cmax is a term used in pharmacokinetics and generally refers to the maximum concentration that a compound or drug achieves after the drug has been administrated.

The term “cannabinoid” as used herein may be understood to refer to a chemical compound that is capable of acting on cannabinoid receptors. Cannabinoids may be produced naturally in the body (endocannabinoids), found in Cannabis plants as well as other plants (phytocannabinoids), or manufactured artificially (synthetic cannabinoids). Cannabinoids produced by the Cannabis plant during its cultivation and growth may include, but is not limited to, cannabigerolic acid (CBGA), tetrahydrocannabinolic acid (THCA), cannabidiolic acid (CBDA), and cannabichromenic acid (CBCA). As used herein, cannabinoid may also refer to the corresponding decarboxylated moieties such as, but not limited to, cannabigerol (CBG), tetrahydrocannabinol (THC), cannabidiol (CBD), and cannabinchromene (CBC) (each of which may be derived from its precursor compound by mild heating typically above 105° C.). Cannabinoids may be synthesized by chemical or biological methods. Phytocannabinoids may be distinguished from endocannabinoids which are chemically distinct, are synthesized in mammalian cells from lipids and other macromolecule precursors, which are not phytocannabinoids, and which may be endogenous ligands of the CB1 and/or CB2 receptors.

The term “dosage form” as used herein may be understood to refer to the physical form of a dose of a chemical compound or formulation used as a drug or medication intended for administration or consumption. Common dosage forms may include pill, tablet, capsule, drink or syrup, aerosol or inhaler, liquid injection, pure powder or solid crystal (e.g., via oral ingestion or freebase smoking), and natural or herbal form such as plant or food of sorts, among many others. The route of administration (ROA) for drug delivery is dependent on the dosage form of the substance.

The term “neurofeedback” as used herein may be understood to refer to a therapy, also referred to as EEG neurofeedback, or EEG biofeedback, that may utilize biofeedback from real-time brain activity to reinforce certain brain function and provide measurements of brain activity. It may be generally understood that neurofeedback may measure brain waves, such as alpha, beta, gamma, delta, and/or gamma waves.

Cannabinoids may be understood to include certain chemical compounds that bind to chemical receptors in the human body. In particular, cannabinoids may bind to specific cellular receptors referred to as cannabinoid receptors. This system in the human body is generally referred to as the endocannabinoid system and may result in the binding of both endogenous and exogenous compounds to cellular cannabinoid receptors. Cannabinoid compounds may be classified into three groups: endocannabinoids, phytocannabinoids, and synthetic cannabinoids. Phytocannabinoids may be understood to include chemical compounds concentrated in the oily resin of plants such as cannabis and hemp. Over 100 phytocannabinoids have been identified, many of which may show beneficial medical properties.

Cannabinoids can be administered to the human body through multiple routes including, but not limited to, oral ingestion, sublingual, buccal, topical, inhalation, rectal, injection, and transdermal absorption. Currently, common practice often includes isolating phytocannabinoid compounds and diluting the isolates in an oily medium that can be consumed or, to avoid the first pass effect, absorbed through buccal or sublingual routes. The isolated phytocannabinoid compounds can be either specifically isolated to obtain a single phytocannabinoid compound (often referred to as single isolate) or be isolated to obtain a plurality of heterogeneous phytocannabinoid compounds (often referred to as whole-plant extract). Single isolated cannabinoids may be advantageous due to the elimination of unwanted compounds such as tetrahydrocannabinol (THC), the psychoactive component. Non-psychoactive cannabinoids, such as cannabidiol (CBD), have no psychoactive side effects and avoid toxicity often encountered with the high doses of THC. Further, these cannabinoids, and others, may be useful in treating certain medical diseases, disclosed elsewhere herein.

While cannabinoids have received most of the attention from researchers, the acidic forms of cannabinoids have shown promise for their medical and physiological benefits. Cannabigerolic acid (CBGA) serves as the common precursor molecule for phytocannabinoids. CBGA may then be converted to additional cannabinoid acids including CBDA, CBCA, THCA, and others. The acids may then be decarboxylated to produce the more common forms of cannabinoids such as cannabidiol (CBD).

Despite being overshadowed by the more common forms of cannabinoids; the acidic forms of cannabinoid acids may have promising therapeutic and preventative characteristics. CB GA may have analgesic, antibacterial, antiviral, anti-inflammatory, and anti-proliferative properties. Additionally, cannabinoid acids may combat cardiovascular disease, metabolic disorders, and colon cancer. Further, cannabinoid acids may have physiological benefits including treatment of inflammation, anxiety, seizures, cancers, pain, and nausea. The cannabinoid acids may also demonstrate anti-bacterial and anti-viral properties as well.

One aspect of the embodiments disclosed herein includes a formulation comprising a cannabinoid acid. In certain embodiments disclosed herein, the cannabinoid acid may be an active ingredient.

The formulation may include one or more cannabinoid acids. In one embodiment, the cannabinoid acid may be CBGA. In another embodiment, the cannabinoid acid may be CBDA. In yet another embodiment, the cannabinoid acid may be CBCA. In another embodiment, the cannabinoid acid may be THCA. In another embodiment, the cannabinoid acid may be CBNA. And in yet another embodiment, the cannabinoid acid may be CBLA. In yet another embodiment, the cannabinoid acid may be a combination of one or more of CBGA, CBDA, CBCA, CBNA, CBLA, and THCA.

In some embodiments, the cannabinoid acid or combination of cannabinoid acids may be the only active ingredient in the formulation.

In certain embodiments, the formulation may include highly-enriched concentrations of a cannabinoid acid or combination of cannabinoid acids.

In an embodiment of the first aspect, a cannabinoid acid may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In one embodiment of the first aspect, a combination of two or more cannabinoid acids may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBDA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBGA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBCA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBNA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, CBLA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

In an embodiment of the first aspect, a combination of CBDA and CBGA may comprise at least 10% by weight of the formulation, at least 20% by weight of the formulation, at least 30% by weight of the formulation, at least 40% by weight of the formulation, at least 50% by weight of the formulation, at least 60% by weight of the formulation, at least 70% by weight of the formulation, at least 80% by weight of the formulation, at least 90% by weight of the formulation, at least 95% by weight of the formulation, at least 98% by weight of the formulation, at least 99% by weight of the formulation.

It will readily apparent that other combinations of cannabinoid acids may also be included in the following disclosure as exemplified above, though not explicitly disclosed herein. Such disclosure may be duplicative and will be appreciated by one of skill in the art to include any combination of CB GA, CBDA, CBCA, CBNA, CBLA, and THCA.

In one embodiment the formulation is substantially free from impurities. As used herein, “substantially free of impurities” shall be understood to refer to impurities, including any cannabinoid and/or cannabinoid acid not intended to be present in an amount by weight of the formulation of greater than about 10%, greater than about 5%, greater than about 1%, greater than about 0.1%, or greater than about 0.01%.

In a further embodiment, the formulation is substantially free from other cannabinoids and/or cannabinoid acids. As used herein, “substantially free from other cannabinoids and/or cannabinoid acids” shall be understood to refer to other cannabinoids and/or cannabinoid acids, including any cannabinoid or cannabinoid acid not intended to be present in an amount by weight of the composition greater than about 1%, greater than about 0.1%, or greater than about 0.01%.

In some embodiments as disclosed herein, the formulation may include one or more dietary supplements including but not limited to antioxidants, Vitamin A, beta-carotene, biotin, boron, Vitamin B (including Vitamin B1, B2, B3, B5, B6, B7, B9, and B12), calcium, cesium, choline, chromium, cooper, Vitamin C, Vitamin D, Vitamin E, folate, folic acid, Vitamin K, magnesium, magnesium oxide, manganese, molybdenum, niacin, nitric oxide, potassium, riboflavin, selenium, thiamin, zinc, and zinc gluconate, and combinations thereof. In certain embodiments, plant extracts may be included other than those extracts from hemp or cannabis, including but not limited to Moringa leaf extract, acai, cesium, echinacea, elderberry, primrose, flaxseed, garlic, ginger, ginkgo, ginseng, grape seed extract, green tea, guar gum, sage, peppermint oil, pomegranate, rhodiola, turmeric, and combinations thereof.

In addition to certain supplements as disclosed above, or being provided without supplements, certain embodiments of the formulation may include a cannabinoid acid and a preservative. The preservative may include one or more of antioxidants, such as Vitamin C, Vitamin E, butylatedhydroxyanisole, butylatedhydroxytoluene, propyl gallate, and the like; antimicrobial agents, such as phenol, parabens, aryl and alkyl acids, and the like. In some embodiments, the formulations may include a combination of the preservatives as well as a combination of the preservatives and any combination of other formulations disclosed herein.

It will be understood by those skilled in the art that analogous nomenclature may be used for some or all of the foregoing components. For instance, it is well within the knowledge of one skilled in the art that Vitamin A may also encompass or be referred to herein as retinol and/or retinoic acid, Vitamin C may also encompass, or be referred to herein as ascorbate and/or ascorbic acid, and so forth. Use of one nomenclature in this disclosure should not be construed as limiting.

In certain embodiments, the formulation may include a combination of cannabinoid acid and moringa leaf extract. In certain embodiments, the cannabinoid acid may be a single acid group or combinations thereof. In certain embodiments, the cannabinoid acid may be CBDA or CB GA, or a combination thereof.

In some embodiments, the formulation may include a combination of cannabinoid acid and ascorbic acid (Vitamin C). In certain embodiments, the cannabinoid acid may be a single acid group or combinations thereof. In certain embodiments, the cannabinoid acid may be CBDA or CB GA, or a combination thereof.

In some embodiments, the formulation may include a combination of cannabinoid acid and magnesium oxide. In certain embodiments, the cannabinoid acid may be a single acid group or combinations thereof. In certain embodiments, the cannabinoid acid may be CBDA or CB GA, or a combination thereof.

In some embodiments, the formulation may include a combination of cannabinoid acid and zinc gluconate. In certain embodiments, the cannabinoid acid may be a single acid group or combinations thereof. In certain embodiments, the cannabinoid acid may be CBDA or CBGA, or a combination thereof.

In yet another embodiment, the formulation may include a combination of cannabinoid acid, moringa leaf extract, ascorbic acid, magnesium oxide, and zinc gluconate. In certain embodiments, the cannabinoid acid may be a single acid group or combinations thereof. In certain embodiments, the cannabinoid acid may be CBDA or CBGA, or a combination thereof.

In addition to the foregoing formulations, certain embodiments may include a formulation comprising any of the previously disclosed formulations along with a fatty acid chain molecule. In some embodiments, the fatty acid chain may be a short chain, medium chain, long chain, or very long chain. In certain embodiments, the fatty acid chain may be 1 carbon in length, may be 2 carbons in length, may be 3 carbons in length, may be 4 carbons in length, may be 5 carbons in length, or may be 6 carbons in length. In certain embodiments, the fatty acid chain may be 6 carbons or less. In some embodiments, the fatty acid chain may be at least 8 carbons in length, at least 10 carbons in length, at least 12 carbons in length, at least 14 carbons in length, at least 16 carbons in length, at least 18 carbons in length, at least 20 carbons in length, at least 22 carbons in length, or at least 24 carbons in length.

The linking of the cannabinoid acid to a fatty acid chain may be useful in increasing the bioavailability of the cannabinoid acid(s) in the human body.

In some embodiments as disclosed herein, a delivery system may be disclosed for delivering a formulation comprising at least an acidic form of cannabinoid acid. In some embodiments, the delivery system may be selected from oral, buccal, sublingual, topical, inhalation, rectal, or injection delivery. In certain embodiments, the delivery system may be selected from oral, buccal, or sublingual. In certain embodiments, the delivery system may be a combination of the foregoing.

In some embodiments, the delivery system may be configured to deliver a formulation comprising an acidic form of a cannabinoid acid to a target site. The target site may be generally any site on or within a patient that is identified for therapy, or as a pathway to carry the formulation. For instance, the target site may be blood, blood plasma, lymphatic system, tissue, brain, muscle, and/or locations of cannabinoid receptors. In certain embodiments, the formulation may include components as previously disclosed elsewhere herein. In certain preferred embodiments, the formulation may include at least one cannabinoid acid selected from the group consisting of CBGA, CBDA, CBCA, CBNA, CBLA, and THCA. In some embodiments, the formulation may comprise a combination of the foregoing cannabinoid acids.

In some embodiments, the formulation may include at least one cannabinoid acid, moringa leaf extract, ascorbic acid (Vitamin C), magnesium oxide, and zinc gluconate. In certain embodiments, the cannabinoid acid may be CBGA, CBDA, or a combination thereof. It will be appreciated by those skilled in the art that one or more formulations as disclosed elsewhere herein may be utilized in the delivery system.

The formulations as described elsewhere herein are believed to have increased bioavailability of the active ingredient or active ingredients as compared to more traditional cannabinoid formulations and delivery methods.

In certain embodiments, the delivery system is configured to preferentially transport at least a portion of the formulation to the lymphatic system.

It will be understood that, generally, a formulation when administered to a patient may have varying bioavailability based on the route of administration. A formulation administered intravenously could be 100% if 100% of the formulation reaches blood circulation. In contrast, the percentage of bioavailability may decrease for oral administration because the active ingredient may often be metabolized, at least partially, before reaching systemic blood circulation or its target site.

Generally, CBD may be understood to have poor bioavailability due to factors such as its lipophilicity. CBD tends to not be ionizable at biological pH, especially in saliva for oral administration. The cLogD for oral preparations of CBD may be around 6.3. However, CBDA has a cLogD of approximately 2.8 at a pH of about 7, and therefore may have much better bioavailability for oral administration. Similar to CBDA, other cannabinoid acids may also tend to have better bioavailability then the downstream cannabinoid components.

In one embodiment, the delivery system may be configured such that the delivery of the formulation to a patient may result in the preservation of the acidic form of the cannabinoid acid(s) and achieving a high bioavailability of the cannabinoid acid.

However, despite better bioavailability, cannabinoid acids may still be susceptible to elimination through metabolism and breakdown. To reduce such susceptibilities, incorporation of fatty acid chains as described further herein may direct delivery of the formulation to the lymphatic system. Under such circumstances, the metabolism and breakdown of the cannabinoid acid(s) may be reduced, and the bioavailability may be increased, which may result in better therapeutic outcomes.

In certain embodiments, the inclusion of fatty acid chains may be employed to direct the formulation to the lymphatic system. Medium (6-12 carbons) and long chain (13-21 carbons) fatty acids may be transported into intestinal lymph system. It has been previously demonstrated that Palmitic acid (16:0) was found to transport the highest percentages to the lymph system (59-68%) as compared to linoleic (18:3) 32%, lauric (12:0) 28%, and decanoic (10:0) 13%.

In some embodiments, short chain fatty acids, which are 6 carbons or less, may be employed to direct the formulation to the lymphatic system. Short chain fatty acids may include fatty acids such as formic acid, acetic acid, propionic acid, butyric acid, isobutyric acid, valeric acid, isovaleric acid, or 2-methylbutyric acid. These may also be referred to as formate, acetate, propionate, butyrate, isobutyrate, valerate, isovalerate, and 2-methylbutanoate, respectively.

In some embodiments, the delivery system may result in the absolute bioavailability of the cannabinoid acid(s) after oral dosing of at least 1%, of at least 5%, of at least 10%, of at least 15%, of at least 20%, of at least 25%, of at least 30%, of at least 35%, of at least 40%, of at least 45%, of at least 50%, of at least 55%, of at least 60%, of at least 65%, of at least 70%, of at least 75%, of at least 80%, of at least 85%, of at least 90%, and of at least 95%.

In certain embodiments, the use of fulvic acid may increase the water-solubility of the cannabinoid acid(s). This increased water-solubility may increase the effectiveness of the drug delivery system. Further, the increased water-solubility may increase the bioavailability of the cannabinoid acid(s).

Fulvic acid is a naturally occurring molecule that has water-soluble properties. Fulvic acid may be used for its role in protecting host cells from oxidation due to environmental toxins. Additionally, it may make nutrients and molecules more soluble and available for use by host cells. In certain embodiments, fulvic acid may be paired with cannabinoid acid(s) to increase the solubility and bioavailability. It may be considered that the structure of fulvic acid being more aliphatic and less aromatic than humic acids, while also having more carboxylic acid, phenolic, and ketonic groups may promote the increased water solubility properties of fulvic acid.

In some embodiments, the fulvic acid may act as a chelating agent for the formulation and delivery system such that it may increase the bioavailability and uptake of the formulation ingredients, including, but not limited to the cannabinoid acid(s).

Other water-soluble vehicles for the formulation may also be considered and may be known in the prior art. For example, micro-encapsulation of extract material may be utilized. Reference is made to PCT/US2018/054216 for “Methods of preparing solulizable, encapsulated plant-based compositions, products based on same” which is incorporated herein in its entirety. Further reference is made to PCT/US2019/045517 for “Encapsulated cannabinoid comestible noble metal compositions and products, methods of preparing same” which is incorporated herein in its entirety.

In yet further embodiments, the active agent(s) may be modified. One such modification may be polarization. In some embodiments, Mikaelian Resonance Technology (polarization) may be employed to apply non-invasive, quantum biotechnology to increase the bioactivity of the active ingredient, such as cannabinoid acid(s). Additionally, such technology may also increase the absorption and bioavailability of the active ingredient. The polarization technology may be capable of exciting the electron state of a target molecule so that it more readily engages in biochemical activity. Studies have shown that polarized CBD may be able to increase Cox-2 inhibition in vitro when compared to non-polarized CBD. Additionally, varying levels of polarization may be applied such that medium-intensity and high-intensity molecules can be created.

In some embodiments, a formulation comprising one or more cannabinoid acids may be useful in treating and/or preventing certain diseases or medical conditions.

CBGA may have analgesic, anti-bacterial, anti-viral, anti-inflammatory, and anti-proliferative properties. Additionally, CBGA may combat cardiovascular disease, metabolic disorders, and certain cancers. Additionally, CBDA may have physiological benefits including treatment of inflammation, anxiety, seizures, certain cancers, and nausea. CBDA may also demonstrate anti-bacterial and anti-viral properties.

In one embodiment, a method of treating or preventing viral infections is disclosed. In certain embodiments, a formulation comprising a cannabinoid acid(s) may be used to treat or prevent viral infections through modulating the immune response, the immune system, viral recognition and entry to target cells, viral replication, viral maturation, and/or viral release.

In some embodiments, a method of treatment or prevention is disclosed utilizing immunomodulation pathways, including, but not limited to pro- and anti-inflammatory functions, Cox-2 pathways, t-cell pathways, and/or neutrophil responses. It will be appreciated that other immunomodulation pathways, cell types, and molecules involved in the host immune response may be affected by the disclosed formulations herein and are not to be limited in the scope of this disclosure.

In some embodiments, the formulation comprising at least one cannabinoid acid group may demonstrate immunomodulation effects via the Cox-2 pathway. Cannabinoids are understood to provide anti-inflammatory effects as well as other pharmacological effects that are still undergoing research. Cox-2 is an enzyme involved in inflammation and chronic inflammatory states. Cannabinoid acids may have Cox-2 inhibitory effects which result in decreased inflammation and chronic inflammatory states. Further, cannabinoid acids may have different anti-inflammation pathways when compared to more traditional CBD and THC anti-inflammation pathways. In some aspects, cannabinoid acids may inhibit Cox-2 mediated prostaglandin production, which results in anti-inflammatory effects.

In some embodiments, other anti-inflammatory pathways may be utilized. For example, cannabinoid acids may have favorable regulatory effects on Treg17 cells and neutrophil subtypes that have anti-inflammatory functions. Treatment methods utilizing formulations as disclosed herein may provide reduced cell death, restoration of neutrophil N1 and N2 imbalance, while also preserving Treg17 cells and decreasing Th-17 cells.

Although inflammation is critical to the healing of injured tissue, excessive and/or prolonged inflammation can often exacerbate tissue injury and lead to further complications. Mechanisms contributing to upregulation of inflammatory response have been the focus of intense investigation, however, mechanisms and treatment therapies that contain and resolve inflammation are increasingly being uncovered. In light of this research, cells of innate and adaptive immunity are understood to undergo context-specific polarization. This context-specific polarization may result in functional phenotypes consistent with pro- or anti-inflammatory outcomes for a patient. Neutrophils have been identified as one cellular type to assume pro- and anti-inflammatory functional phenotypes, namely N1 and N2, respectively. T cells have also been identified as undergoing polarization to their suppressive/regulatory phenotypes. In particular, STAT3-dependent T helper 17 (Th-17)-specific regulatory T (Treg17) cells may undergo this polarization.

Treatment of a patient with formulations as described herein, comprising cannabinoid acids, may demonstrate positive effects on the neutrophil and T cell pathways resulting in decreased prolonged inflammation and cell damage. It may be understood that the use of cannabinoid acids as a possible treatment regimen may modulate inflammatory responses leading to better patient outcomes.

In another embodiment, a method of treating or preventing infection of SARS-CoV-2 is disclosed. In certain embodiments, inventors propose a mechanism of action contributing to antiviral activity of SARS-CoV-2 via immunomodulation and specific formulations comprising one or more cannabinoid acids. In some embodiments, the formulation of one or more cannabinoid acids may be more effective against particular variants of SARS-CoV-2.

SARS-CoV-2 is a member of the Coronaviridae family, and is characterized as an enveloped, non-segmented, positive sense RNA virus that includes crown-like spikes on the outer surface. The SARS-CoV-2 virus is known to contain RNA strands that are 29.9 kb long encoding four main structural proteins, the spike, envelope, membrane, and nucleocapsid proteins, 16 nonstructural proteins, and several accessory proteins. Any step of the SARS-CoV-2 virus infection and replication cycle is a potential target for antiviral intervention.

Two cannabinoid acids have shown high affinity for the spike protein of SARS-CoV-2, CBGA and CBDA. In in vitro experiments with human epithelial cell lines suggested that CBGA or CBDA, individually, may reduce infection by a pseudovirus expressing the spike protein.

In some embodiments, formulations comprising cannabinoid acids disclosed herein may be used to treat patients infected with SARS-CoV-2. Additionally, formulations comprising cannabinoid acids disclosed herein may be used to prevent patients from becoming infected with SARS-CoV-2. In some embodiments, the use of formulations as disclosed herein may prevent patients from becoming infected with a sufficient load of SARS-CoV-2 such that no symptoms are identifiable, and transmission of the virus is reduced.

In some embodiments, a formulation as previously disclosed herein is delivered to a patient via oral, buccal, sublingual, topical, inhalation, rectal, or injection delivery.

In some embodiments, a pharmaceutically effective does of a formulation as previously disclosed herein is delivered to a patient for the treatment of SARS-CoV-2. In some embodiments, a pharmaceutically effective does of a formulation as previously disclosed herein is delivered to a patient for the prevention or reduction of infection by SARS-CoV-2 of the patient.

In addition to the foregoing, cannabinoid acids may also be used to manage pain as well as neurological deficits, developmental disorders, and neurodegeneration. Neurological conditions may include sleep disorders, autism, ADHD, epilepsy, Parkinson's disease, dystonia, autism, depression, and dementia.

In particular, the use of cannabinoids, alone, or in combination with other therapies may serve as an effective treatment for chronic pain. Pain perception may be considered as a complex process, whereby the pain perceived by an individual is an integration of current information about the sensory stimulation and prior information from previous experiences which influence the emotions, attention, and expectations of the individual about the pain. Several studies have been performed to identify neurophysiological correlates of chronic pain. The brain is understood to be influenced by biological, psychological, and social factors, and plays a central role in the onset and maintenance of pain. Indeed, the literature regarding chronic pain indicates there is a growing body of evidence that there are structural and functional neurophysiological brain abnormalities in individuals with chronic pain. Individuals with chronic pain evidence patterns of brain activity that may differ from individuals that do not present with chronic pain.

Based on these previous studies, it may be understood that chronic pain may be associated with a relative decrease in alpha brain waves, an increase in beta brain waves, and an increase in theta brain waves on electroencephalogram (EEG) recordings. These correlates of pain have been used to develop brain training protocols which may help patients to increase or decrease their brain activity in the direction associated with pain-relief. In particular, a technique utilized for altering brain activity may be referred to as neurofeedback.

Neurofeedback may generally be understood as a therapy or technique which teaches individuals to self-regulate their brain activity by providing them real-time measurements of their EEG or Functional Magnetic Resonance Imaging (fMRI) signals. Neurofeedback may be useful in reducing the severity of neuropsychiatric conditions such as ADHD, depression, anxiety, stroke, and others.

In certain embodiments, neurofeedback may be combined with formulations disclosed elsewhere herein comprising cannabinoid acids to provide treatments for patients suffering from pain. In some embodiments, the formulation may include a fatty acid and cannabinoid acid. In certain embodiments, the fatty acid may be a short-chain, medium-chain, long-chain, or very-long-chain fatty acid. In yet other embodiments, the fatty acid may be a medium-chain fatty acid, In yet another embodiment, the fatty acid may be a short-chain fatty acid. Each of the prior examples of fatty acids may be part of a triglyceride and it will be generally understood that when referring to a fatty acid chain, the fatty acid chain may be a part of a triglyceride.

In some embodiments, a method for treating pain of a patient may include a formulation having a triglyceride and an active ingredient comprising a cannabinoid acid. In some embodiments, the cannabinoid acid may be CBD, CBG, THC, CBN, or combinations thereof. The use of a triglyceride CBD formulation has been found to increase alpha neurofeedback in some patients with chronic pain. In particular, such treatment has been found to have no dose toxicity, fast absorption rates, and therefore has an advantageous effect on dwell time and overall ability to transition patients to higher alpha wave frequencies. Generally, higher alpha wave frequencies are associated with reduced pain.

In one embodiment, a short chain triglyceride CBD formulation with a dosage of 50 mg/mL, 2× per day may increase neurofeedback in certain alpha state parameters including transitions from low to high alpha state, dwell time, and distribution and transition probability. Additionally, the formulation may further include CBG for increased potency in anti-inflammatory, oxidative stress, and neuroinflammation. In some embodiments, the formulation may comprise THC for increased potency in anti-inflammatory and muscle relaxation. The formulation may include CBN for increased potency in mood relaxation and stress relief.

In some embodiments, the dosage form may be up to 1000 mg/oz. In at least one embodiment, the dosage form may be from 1000 mg/oz to 5000 mg/oz. In at least one embodiment, the dosage form may be about 1500 mg/oz. In at least one embodiment, the dosage form may be about 2000 mg/oz. In at least one embodiment, the dosage form may be at least 2500 mg/oz. In at least one embodiment, the dosage form may be about 3000 mg/oz. In at least one embodiment, the dosage form may be about 3500 mg/oz. In at least one embodiment, the dosage form may be about 4000 mg/oz. In at least one embodiment, the dosage form may be about 4500 mg/oz. In at least one embodiment, the dosage form may be about 5000 mg/oz. In some embodiments, the dosage form may be taken 1× a day, 2× a day, 3× a day, or 4× a day.

It has been determined that in one embodiment, use of triglyceride CBD formulation and neurofeedback produces a reduction in pain and change in neurophysiological signals of a patient. In one study, alpha neurofeedback alone yielded a slight increase in alpha waves and lower reported pain. In patients provided with a dosage form of 1500 mg/oz taken lx per day and alpha neurofeedback, patients demonstrated an increase in alpha waves and lower reported pain. In particular, some patients demonstrated an increase in alpha waves over 8 Hz. In some patients, a dosage form of short-chain triglyceride CBD at 1500 mg/oz 1× a day and neurofeedback yielded alpha waves above 8 Hz, lower reported pain, and produced a longer duration in alpha state than those who received the same dosage form, but with medium-chain triglyceride CBD and neurofeedback, or neurofeedback alone. Additionally, patients who received a dosage form of short-chain triglyceride CBD at 3000 mg/oz 1× a day and neurofeedback yielded increased alpha waves of at least 10 Hz, lower reported pain with greater significance and in faster time compared to the 1500 mg/oz patients and had even longer duration of the alpha state. Patients who received the same dosage form of short-chain triglycerides CBD at 3000 mg/oz, but at a higher dose rate of 4× a day, along with neurofeedback, yielded similar results as other dosage forms at 3000 mg/oz at 1× a day, thus, it was determined that the CBD formulation used reached a specific threshold at certain dosages.

Additional studies have demonstrated similar results. Formulations including CBD were administered to patients with chronic pain at 50 mg/mL (3,000 mg/oz) 2× per day. Dosages were given for 3 months. At the end of the study, 90% of patients reported significant reduction of pain and overall increased quality of life. Additionally, 10% reported some reduction of pain and increased quality of life. No side effects were reported in 100% patients.

Thus, it has been shown that cannabinoid acids within certain formulations may enhance neurofeedback and alpha-entrainment which, in turn, may further contribute to pain management through neuroplasticity. Novel neuromodulatory therapies are disclosed where individuals are given real-time feedback regarding their brain neurophysiological signals along with cannabinoid formulations. This therapeutic avenue may promote modulation of pain-associated brain activity. The use of formulations with cannabinoid acids may have the ability to influence the brain mechanisms and thus, allow the brain to resignal, rewire, and compensate for neuronal misconnections overall, which may be associated with chronic pain in some individuals.

Thus, although there have been described particular embodiments of the present invention of new and useful CANNABINOID ACID FORMULATIONS, DELIVERY SYSTEMS, AND METHODS OF USE, it is not intended that such references be construed as limitations upon the scope of this invention except as set forth in the following claim.

What is claimed is:

1. A method of decreasing pain in a patient, the method comprising:
providing a formulation having a triglyceride and an active ingredient comprising a cannabinoid acid;
delivering the formulation to the patient via a delivery system wherein at least 10% of the cannabinoid acid is preserved in its acidic form at a target site; and
performing neurofeedback wherein alpha brain waves of the patient are increased in amplitude.

2. The method of claim 1, wherein the delivery system preferentially transports at least a portion of the formulation to the lymphatic system.

3. The method of claim 2, wherein the delivery system is selected from oral, buccal, or sublingual.

4. The method of claim 1, wherein the triglyceride comprises a short chain fatty acid.

5. The method of claim 1, wherein the neurofeedback is performed in real-time to provide biofeedback to the patient.

6. The method of claim 6, wherein alpha brain waves of the patient increase above 8 Hz during neurofeedback.

7. The method of claim 6, wherein the alpha brain waves of the patient increase above 10 Hz during neurofeedback.

8. The method of claim 1, wherein the cannabinoid acid is CBD.

9. The method of claim 8, wherein the formulation has a dosage form of 1500 mg/oz taken one time per day.

10. The method of claim 8, wherein the formulation has a dosage form of 3000 mg/oz taken one time per day.

11. The method of claim 8, wherein the formulation has a dosage form of 3000 mg/oz taken four times per day.

12. A method of decreasing chronic pain in a patient, the method comprising:
providing a composition having a triglyceride comprising a short-chain fatty acid, an active ingredient comprising a cannabinoid acid wherein the cannabinoid acid is selected from the group consisting of CBDA, CBGA, and CBCA;
delivering the formulation to the patient via a delivery system wherein at least 50% of the cannabinoid acid is preserved in its acidic form at a target site; and
performing neurofeedback wherein alpha brain waves of the patient are increased in amplitude.

13. The method of claim 12, wherein the delivery system preferentially transports at least a portion of the formulation to the lymphatic system.

14. The method of claim 13, wherein the delivery system is selected from oral, buccal, or sublingual.

15. The method of claim 12, wherein the triglyceride comprises a short chain fatty acid.

16. The method of claim 12, wherein the neurofeedback is performed in real-time to provide biofeedback to the patient.

17. The method of claim 16, wherein alpha brain waves of the patient increase above 8 Hz during neurofeedback.

18. The method of claim 12, wherein the formulation has a dosage form of 1500 mg/oz taken one time per day.

19. The method of claim 12, wherein the formulation has a dosage form of 3000 mg/oz taken one time per day.

20. The method of claim 12, wherein the formulation has a dosage form of 3000 mg/oz taken four times per day.
